(19) United States
(12) Patent Application Publication
(10) Pub. No.: US 2005/0107716 A1
(43) Pub. Date: May 19, 2005

(54) Methods and apparatus for positioning and retrieving information from a plurality of brain activity sensors

(75) Inventors: Scott Michael Eaton, Spokane, WA (US); Gary Mc Darby, Dublin 3 (IE)

Correspondence Address: CHARLES G. CALL, 68 HORSE POND ROAD, WEST YARMOUTH, MA, 02673-2516, US

(73) Assignee: Media Lab Europe, Dublin 3 (IE)

(21) Appl. No.: 10/713,440

(22) Filed: Nov. 14, 2003

(51) Int. Cl. A61B 5/04

(52) U.S. Cl. 600/544; 128/903

(57) ABSTRACT

A system for acquiring brain activity data that includes a combined mechanical support frame and signal transmission network structure that consists of a plurality of intersecting, mechanically connected rails, each of which houses electrical signal and power distribution conductors. The conductors in the rails interconnect a variety of different functional node devices that are mechanically supported at adjustable positions on the rails. Each of the nodes houses functional components and include data nodes for acquiring brain activity data, a host node for receiving data from the data nodes and relaying information to an external device, a power node for supplying operating power to the other nodes. The position of the rails may be adjusted relative to each other, and the position of each node may adjusted on the rail upon which it is mounted. The data nodes include retractable probes which may be moved toward or away from the head to position electrodes or other sensors at precise desired locations on the head.

### FIELD OF THE INVENTION

This invention relates to structures and methods for acquiring data indicating brain activity data and/or related physiological and environmental information.

### BACKGROUND OF THE INVENTION

Electroencephalography (EEG) is a widely used brain activity measurement that employs electronic sensing electrodes to measure and record electrical activity in the brain. EEG is a key tool in the diagnosis and management of epilepsy and other seizure disorders and is widely used to assist in the diagnosis of brain damage and disease (e.g., stroke, tumors, encephalitis), mental retardation, sleep disorders, degenerative diseases such as Alzheimer's disease and Parkinson's disease, and certain mental disorders (e.g., alcoholism, schizophrenia, autism). EEG may also be used to monitor brain activity during surgery and to determine brain death, and for other purposes.

In typical applications, before EEG data can be captured, a nurse or technician attaches numerous electrodes to the patient's scalp with a conductive, electrolytic paste. Each electrode is attached to an EEG data utilization device (e.g. an electroencephalograph machine) by long conductive leads and, to avoid disturbing the electrodes and leads, the patient normally lies on a bed, padded table, or comfortable chair and is asked to relax and remain still during the EEG testing period, which may last for nearly an hour. During the test procedure, the patient may be asked to breathe slowly or quickly, and visual stimuli such as flashing lights or a patterned board may be used to stimulate certain types of brain activity. Throughout the procedure, the electroencephalograph machine makes a continuous graphic record of the patient's brain activity, or brainwaves, on a long strip of recording paper or on a computer screen. This graphic record is called an electroencephalogram.

The sleep EEG uses the same equipment and procedures as a regular EEG. Patients undergoing a sleep EEG are encouraged to fall asleep completely rather than just relax. They are typically provided a bed and a quiet room conducive to sleep. A sleep EEG lasts up to three hours, during which time the patient may move about during sleep, possibly disturbing the electrode connections. In an ambulatory EEG, patients are typically hooked up to a portable cassette recorder and may then go about their normal activities, take their normal rest and sleep for a period of up to 24 hours. Ambulatory electroencephalography (AEEG) monitoring requires that the electrode contacts remain secure for prolonged EEG recording when the patient is active, exacerbating the problem of maintaining multiple secure electrode connections to the patient's skin.

EEG recording has proven to be more problematic because of the need to maintain good contact using a multiplicity of electrodes, each of which require signal amplification and multichannel recording. Preamplifiers may be associated with each electrode, but complicate the problem of electrode placement and maintenance for extended durations.

Other useful methods of capturing brain activity data include diffuse optical tomography (DOT) in which tissue is illuminated by near-infrared light from an array of emitters and the light which is scattered through the tissue is measured by an array of photo-sensitive detectors on the surface of the tissue. The technology allows hemodynamic changes resulting from brain activity to be assessed effectively and non-intrusively, but again requires the accurate placement and maintenance of sensors at desired locations at or near the skin surface.

There is accordingly a need for improved methods and apparatus for acquiring brain activity data by accurately placing electrodes, such as electroencephalography and electrocorticography electrodes, and diffuse optical tomography sensors, or a combination of these, at precise locations on human head.

In addition, there is a need for improved methods for transmitting sensed brain signals and brain activity data from a multiplicity of possibly diverse sensors to a data utilization device.

There is a further need for improved methods for accurately positioning and maintaining the position of brain activity sensors on the head in a manner that is readily adjustable, yet mechanically stable and comfortable for the user.

There is a further need for improved, noise free communication of brain activity data and other information to a data utilization device from a multiplicity of separate sensors.

There is a further need for a brain sensor positioning system that may be readily adapted for use with a variety of different sensing and data utilization mechanisms, such as EEG and DOT sensors and a variety of ancillary signal processing and signal responsive components, and which includes mechanisms for providing operating power to these sensors and components as well as providing data pathways between at least selected ones of these devices and local or remote data utilization devices.

There is a still further need for an improved way of placing electrodes at very specific locations on or near the human head. For example, specific places have been identified where evoked potentials associated with attention manifest themselves. Also every human head is different and, as a consequence, a system capable of placing electodes at different locations is required to accommodate individual measurements.

### SUMMARY OF THE INVENTION

In its preferred form, the present invention takes the form of a system for acquiring of data indicating brain activity from a plurality of sensors accurately positioned at desired locations on a human head. The system employs a modular mechanical structure which positions the sensors and further provides a power and signal distribution network which interconnects the sensors with other functional units. The structure consists of a plurality of intersecting, mechanically connected rails, each of which houses exposed electrical signal distribution conductors that extend along substantially the entire length of each rail, the conductors in all of the rails being electrically connected together to form a data and power distribution network. Mechanical nodes connect rail segments together, providing both mechanical and electrical connections between the rails. Each of the intersecting rails are curved such that the resulting support structure defines an anatomically shaped cavity (helmet) that surrounds a human head. The rails are preferably constructed of a light-weight, electrically conductive material such as aluminum to provide shielding for the electrical network conductors.

This structure supports and interconnects a plurality of interoperating nodes, each of which is a separate functional component having a mechanical housing adjustably attached to one of the support rails. Each node interfaces with the system over the power and communications bus provided by the mounting frame. The functional nodes may include: (1) a power supply node for delivering electrical energy to the other nodes, (2) one or more data acquisition nodes for acquiring data indicating brain activity or other information from a localized area on the wearer's head, (3) a host node for receiving data from the data node(s) and relaying the data to an external utilization device, and (4) one or more reference nodes which provide connections to the user's body at the appropriate points to obtain reference and ground potentials.

Each of the node is mounted for sliding movement along its supporting rail. Mechanical nodes which structurally connect the rails together may be moved to reposition the rails relative to one another. Individual nodes may be adjustably moved along their supporting rails. In this way, the rails and the nodes may be repositioned to place each functional node over a desired area of the head. In addition, the data nodes support movable sensing probes that can be adjustably moved toward or away from the head. A probe may be spring-loaded to facilitate a biopotential contact sensor on the distal end of the probe against the skin to insure good contact.

Each data node preferably typically includes a transducer and electronic signal conditioning circuitry, such as an on-board amplifier and analog-to-digital converter for processing sensed EEG signals. By positioning the conditioning circuitry close to the tranducer, preferably less than ten millimeters apart, the extent to which ambient electrical noise can affect the integrity of the sensed signal value is greatly reduced. The transmission of the signal values in digital form along the shielded pathways provided by the distribution rails helps insure that the transmission of the sensed data will be immune from the effects of noise. The host node may include a transceiver which preferably conforms to the Bluetooth specification for transmitting acquired data signals in digital form to the external utilization device.

The data nodes may be used to position a variety of different kinds of sensors at desired locations on the user's head. A data node may deploy a sensor for acquiring a variety of different brain activity signals, such as electroencephalography, electrocorticography, or optical tomography imaging data. In many applications, different kinds of data nodes may be used to concurrently acquire different kinds of brain activity data, as well as additional physiological and environmental data such as body temperature, ambient temperature and pressure, heart and respiration rates, and body reference potentials.

These and other features of the invention may be more clearly understood by considering the following detailed description in which frequent reference will be made to the attached drawings.

### BRIEF DESCRIPTION OF THE DRAWINGS

In the detailed description which follows, frequent reference will be made to the attached drawings, in which:

FIG. 1 is perspective view of a modular brain activity sensing system embodying the invention;

FIG. 2 is a side elevation view of the system shown placed on a human head;

FIG. 3 is a perspective, exploded view of a mechanical node for connecting lateral distribution rail segments to medial rail segments;

FIG. 4 is top elevation view of the mechanical node shown in FIG. 3 interfacing with a medially placed data node;

FIG. 5 is a plan view of a rail segment showing the conductive traces used to transmit power and data signal between nodes in the system;

FIG. 6 is a perspective view of an assembled mechanical node;

FIG. 7 is an exploded view of an EEG data node;

FIG. 8 is a plan view of an assembled EEG data node showing internal elements with dashed lines;

FIG. 9 is a perspective view of an assembled reference node;

FIG. 10 is an exploded view of a host node with integrated battery housings; and

FIG. 11 is a block diagram illustrating how the various nodes which make up an illustrative system are interconnected by the power and signal busses provided by the distribution rails.

### DETAILED DESCRIPTION

The preferred embodiment of the invention is a self-contained, modular, wireless, head-mounted brain activity sensing system suitable for clinical, research, and interface applications. The invention can be used to advantage for the acquisition of brain imaging and sensing data. The preferred embodiment of the invention consists of a modular mechanical frame which houses a power and communications bus to which a plurality of sensors and other devices can be attached. The sensors are designed for brain signal acquisition and other sensing tasks. A host controller residing on the same bus coordinates the signal acquisition and outputs the data to a wireless link for remote processing.

The system is made up of a collection of functional blocks, each of which implements a subset of the overall system functionality. As used herein, the term “node” refers to a functional component that has a mechanical housing and may interface with the system over the power and communications bus provided by the mounting frame. In the preferred embodiment, there are six classes of nodes: host nodes, power nodes, distribution nodes, reference nodes, data nodes, and mechanical nodes. These nodes slide onto distribution rails that provide both a mechanical structure for positioning the nodes and a housing for the power and communications bus that links the nodes.

The system is built around object-oriented design patterns, with each node type encapsulating specific functionality independent of the function of the other nodes. Briefly, a host node is responsible for mastering the serial communications bus, controlling input and output from the system, and monitoring system performance. A power node provides a well regulated power supply to the system. Distribution nodes serve to interconnect the signal and power tracks from the medial distribution rails to lateral distribution rails. Reference nodes connect the system's reference and ground signal tracks to the user's body at the appropriate points. Data nodes house the transducer circuitry and provide the sensing functionality to the system. Finally, mechanical nodes act as structural supports, positioning and maintaining a comfortable fit on the head.

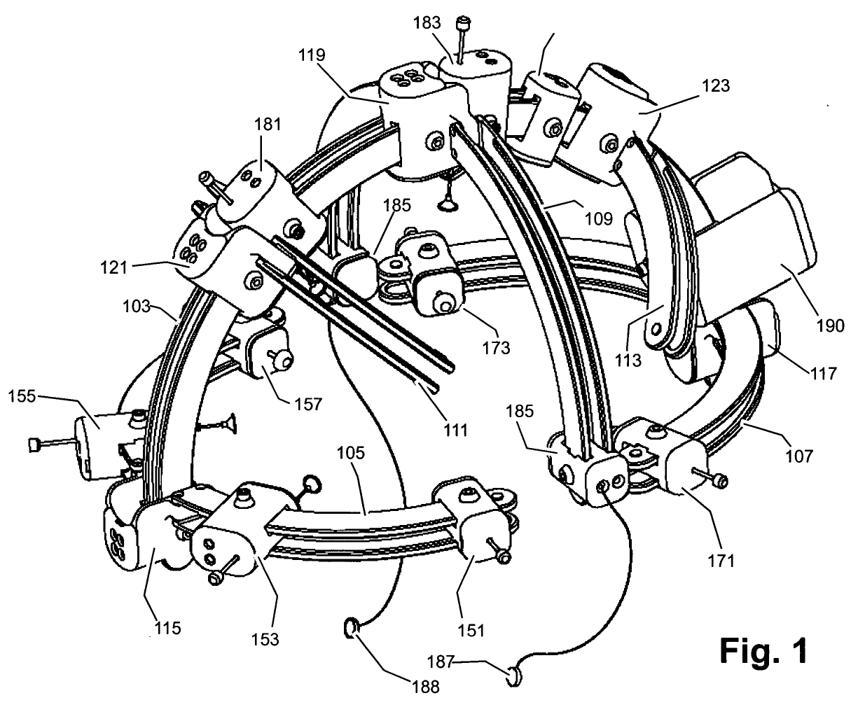

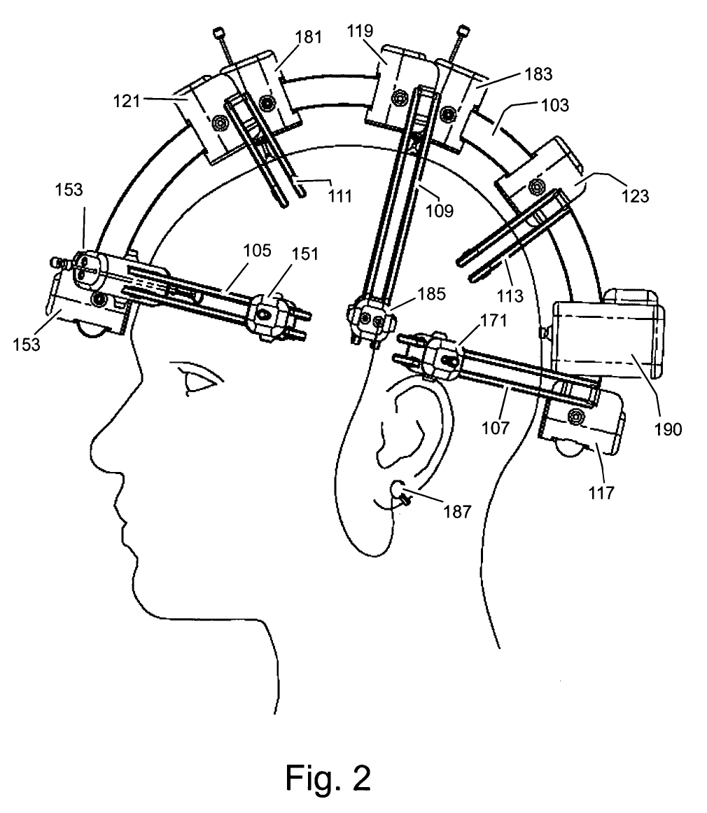

An illustrative example of the preferred embodiment of the invention is shown in FIG. 1. A pair of parallel, curved distribution rails seen at 103 forms a medial arch within a node support frame that defines an anatomically-shaped “helmet” to be worn over a human head as seen in FIG. 2. Longer pairs of lateral distribution rails are attached to near the respective ends of the medial rail 103 as seen at 105 and 107. Longer pairs of lateral distribution rails seen at 109 are attached to the medial rails 103 near their midpoint. Two pairs of shorter lateral rails seen at 111 and 113 are also attached to the medial rails 103 to complete the skeleton of the support frame. These five rails, in combination with the medial rail, allow electrode placements to conform to the 10-20 standard.

The lateral rails 105, 107, 109, 111 and 113 are mechanically secured to the medial rails 103 by distribution nodes 115, 117, 119, 121 and 123 respectively. The distribution nodes provide both mechanical and electrical connections between the rails. The resulting skeleton provided by the distribution nodes and rails provides both mechanical support and electrical connections between the remaining nodes which make up the system. In the illustrative embodiment seen in FIG. 1, the lateral rails 105 support two data nodes seen at 153, 155 and two mechanical nodes 151, 157; two mechanical nodes 171 and 173 are supported at the ends of the lateral rails 107; and two data nodes 181 and 183 are supported on the medial rails 103. Reference nodes 185 and 186 are supported at the ends of the lateral rails 109. Electrically conductive wires connect contact pads 187 and 188 to the reference nodes 186 and 187 respectively. The contact pads 187 and 188 are attached to the skin at the wearer's ears as seen in FIG. 2 and provide connections between the user's body and the ground and reference tracks (to be described) in the support rails. A host node 190 for the system is supported on the medial rail 103.

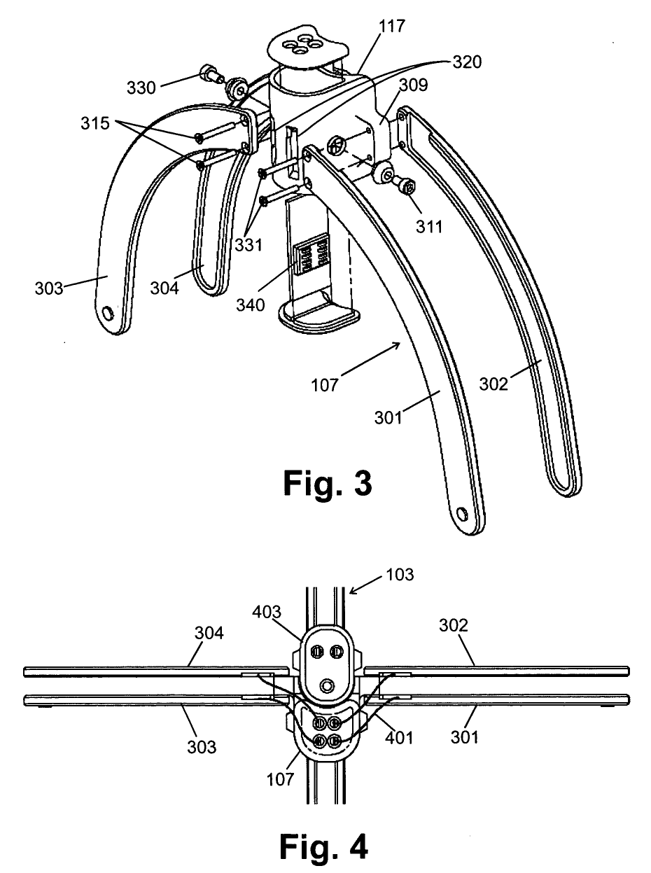

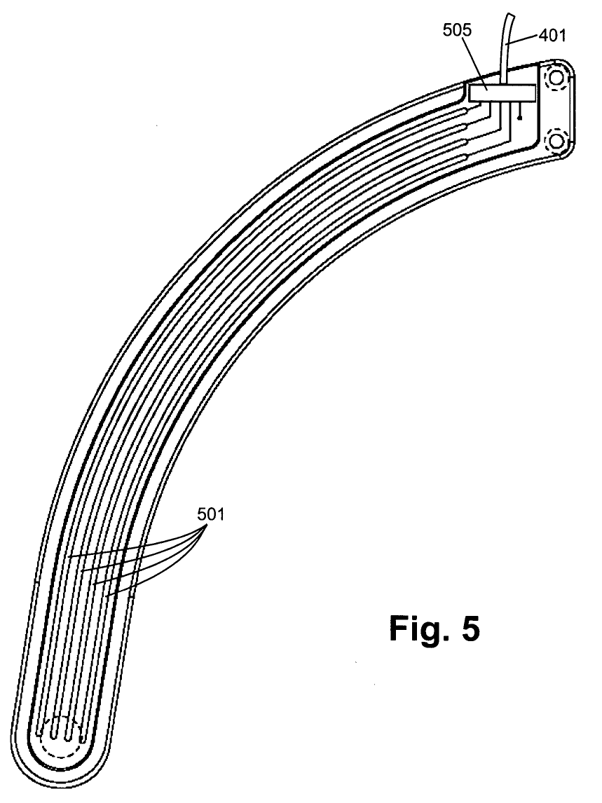

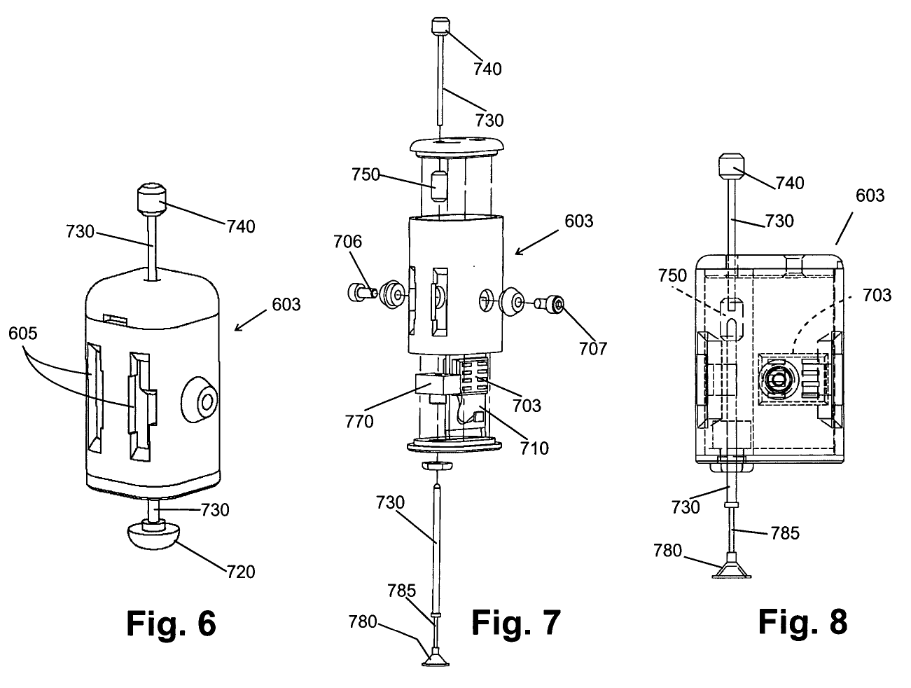

The manner in which the distribution nodes connect the lateral and medial distribution rails may be seen more clearly in FIG. 3 which shows how the distribution node 117 is attached to the four separate rail segments 301-304 which together form the lateral distribution rail 107. A flange as seen at 309 extends outwardly from the housing of distribution node 117 and the two rail segments 301 and 302 are secured to the flange 309 by the fasteners 311. Similarly, the rail segments 303 and 304 are secured by fasteners 315 to a flange (not seen) that extends outwardly from the opposing side of the housing of distribution node 117. The medial rails 103 (not shown in FIG. 3) pass through slots 320 through the housing of node 117. Set screws seen at 330 and 331 secure the node to the medial rails 103. When the set screws 330 and 331 are loosened, the distribution node 117 and the lateral distribution rail 107 which it supports may be repositioned with respect to the medial rail 103.

The lateral and medial rail segments provide a mechanical support for other nodes used by the system and also provide these nodes with connections to power and data communications networks. The power and data lines in the medial and lateral rails are connected at the distribution node 117 by cross connect lines, one of which is seen in FIG. 4 at 401. As seen in FIG. 5, one face of each rail exposes four parallel conductive traces seen at 501 which carry electrical power and data signals. Since two rail segments pass through each node, a total of eight electrical conductors are available for power and signal distribution between the nodes. At the end of the rail segment which is secured to a distribution node, the wire traces 501 are connected to respective conductors within a four-wire cross-connect line 401 (also seen in FIG. 4) at a connector block 505. As seen in FIG. 3 at 340, an array of contact pads within the housing of the mechanical node 117 provides sliding electrical connections between the cross-connection lines and the corresponding exposed wire traces in the medial distribution rails. In this way, all of the wire traces in both the lateral and medial distribution rail segments are connected together and form a common eight-wire bus for interconnecting all of the nodes mounted on the support frame.

Like the distribution node seen at 117, all of the other nodes are mounted for sliding movement along the support rails when their set screws are loosened. The array of contact pads are spring loaded and immediately establish contact with the conductive tracks when the node is inserted onto the rails. The set screw only secures the node in-place by locking friction with the outside of the rail. The set screws are tightened after the node has been positioned as desired on the supporting rails to secure the node in the selected location. The housing of the distribution node is shaped such that another node, such as the data node seen at 403 in FIG. 4, may be positioned adjacent to the intersection of the rails. The interface between the EEG data node and the distribution node allows for the medially placed electrode to fall inline with the electrodes of the lateral EEG nodes, thereby conforming to the 10-20 system.

The EEG data nodes, four of which are seen at 153, 155, 181 and 183 in FIG. 1, are constructed as shown in FIGS. 7-8. Each node consists of components mounted within a housing seen generally at 603. Each node is supported by a pair of support rails which are inserted through rectangular openings 805 in the sidewalls of the housing 603. Two contact pads, one of which is seen at 703 in FIGS. 7 and 8, each contain an array of spring-loaded electrical contacts that engage with and establish electrical connections to the exposed conductive traces (seen at 501 in FIG. 5) in the supporting rails that pass through the housing 603. Set screws seen at 706 and 707 are tightened to mechanically secure the housing 603 in place on the support rail.

The contact pads are mounted on the surfaces of a circuit support board 710 which also supports additional electronic circuitry (not shown) within the node housing, such as an integrated circuit for amplifying and digitizing signals picked up by a biopotential electrode 780 positioned at the distal end of a preferably gold-plated, electrically conductive probe 730. A control knob 740 attaches to a threaded mechanical shaft 730TOP and is attached to the conductive probe 730 by a non-conductive coupling 750. The knob 740 may be grasped by the hand, permitting the electrode 780 to be moved longitudinally toward or away from the user's head and may also be twisted to obtain a good electrical contact with the skin. The probe 730 makes sliding electrical contact with a connector jack 770. The probe 730 as shown in FIG. 7,8 deploys an biopotential electrode 780 at the end of an extension member 785 which telescopes inside the hollow tube of the probe 730. A spring (not shown) within the probe 730 applies resilient pressure to the extension 785 to urge the electrical contact 780 against the skin.

A reference node is shown in FIG. 9. Like the data nodes, the reference node housing 903 is provided with rectangular openings 806 in its sidewalls to receive the supporting rails. A set screw 910 is tightened to secure the reference node in place. Two connector ports at 920 are included to permit the connection of lead wires which extend to contact electrodes (as seen at 187 and 188 in FIG. 1).

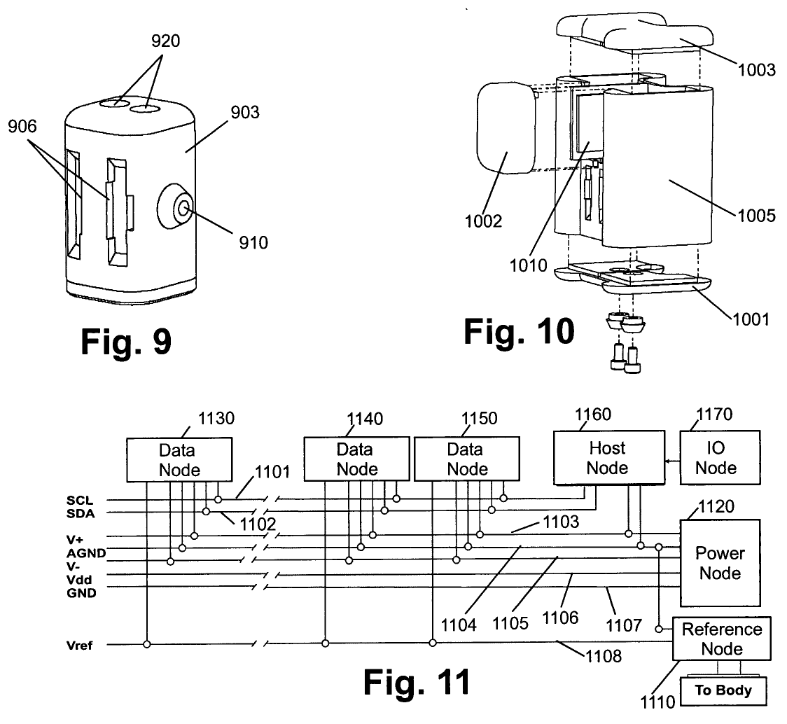

FIG. 10 illustrates the construction of a host node. Removable covers seen at 1001, 1002 and 103 may be detached from the node housing 1005 to permit access to the internal electronics within the unit which typically consists of one or more integrated circuits mounted on a circuit board seen at 1010. The integrated circuits preferably include a microcontroller for transmitting control signals to the nodes and providing bus mastering communication control which permits a large number of nodes to communicate over a single shared data bus. The microcontroller may communicate with external devices via a short range radio transceiver which is compliant with the Bluetooth protocol for providing short range communications between the host node and an external utilization device. The host unit may include a power supply, or power may be provided by a separate power node (not shown). Similarly, the host unit may include its own communications module, or may communicate with an external utilization device via a separate IO module that interfaces with connectors on the host node (not show). The Bluetooth protocol is described in detail in the Specification of the Bluetooth System, Volume 1, Version 1.1 (Feb. 22, 2001) available at www.bluetooth.org. Bluetooth compliant transceiver may be implemented with existing integrated circuit products, such as the Motorola MC71000 Bluetooth Baseband Controller, the Motorola MC113180 Bluetooth RF Transciever Integrated Circuit, the Motorola MRFIC2408 External Power Amplifier IC, and the Motorola MC13181 power management chip.

The manner in which the various nodes that make up the system are interconnected to the power and data distribution network is illustrated in the block diagram of FIG. 11. The distribution network in the example of FIG. 11 consists of a serial clock line (SCL) at 1101, a serial data line (SDA) at 1102, a positive analog supply conductor (V+) at 1103, an analog ground line (AGND) at 1104, a negative analog supply conductor (V−) at 1105, a digital supply voltage (Vdd) 1106, and a signal ground (GND) 1107, and a reference voltage line (Vref) at 1108. The ground (AGND) and reference lines 1104 and 1108 are both connected to the human body via the reference node shown at 1110. A power node 1120 provides power to the power bus comprising conductors 1103, 1104 and 1105. The data and control lines 1101 and 1102 connect each data node (1130, 1140 and 1150) to the host node seen at 1160 which includes, or is connected to, an IO module 1170 that preferably includes a Bluetooth transceiver for communicating data with an external utilization device.

To realize a working system, a single power node, a single host node, and at least one data node must be interfaced with the distribution rails (an additional reference node is required if bioelectric signals are being recorded). Though only a single data node is needed to create a working system, the system supports a plurality of data nodes with the number limited only by the ability of the host node to master serial communications to all nodes at the desired sampling rate, the ability of the power supply to source enough current, and spatial constraints of the nodes themselves.

The object-oriented system design is a central feature of the invention. This modular approach results in an extremely flexible platform for brain research and interface development. Because the functionality of each data node is entirely independent of the configuration of the skeleton or the other data nodes in the system, diverse types of sensors can be used simultaneously. Any technology can be incorporated into the system by way of a data node given that it implements the power and communications interface to the system and can be sufficiently miniaturized so as to be head mounted (or body-mounted with a lead to an data node on the bus). The preferred embodiment of the invention uses electroencephalography (EEG) data nodes which will be described in detail later. Other useful types of data nodes include: diffuse optical tomography (DOT) emitters and receivers for imaging the distribution of chromophores in the cortex, nodes for interfacing with electrocorticography (ECoG) implanted electrodes, location sensors for patient tracking, transducers for audio or video, and other nascent technologies.

The flexibility of the power and communications bus allows the user to easily combine two or more sensing technologies simultaneously. For example, by combining EEG data nodes with DOT data nodes, it is possible to maximize the benefits of each modality while minimizing the overall limitations of the system. In this case, EEG nodes give the system high temporal resolution but limited spatial resolution. DOT nodes complement this by providing improved spatial resolution, though they lack the temporal resolution of the EEG nodes. Other data nodes could be added to provide other application-specific information (patient location, environmental noise, temperature, etc).

To properly interface with the system, data nodes are responsible for digitizing their samples and passing this data over the serial bus on command. The serial bus is mastered by a host node that controls all serial communications on the bus. The host node collects the samples from all active data nodes and packages the acquired data for transmission to an input/output (IO) module for onward processing. The implementation of the IO module is application dependent and, in keeping with the object-oriented nature of the system, is entirely reconfigurable. The module can take the form of: a wireless link (Bluetooth, Wi-Fi, or other) for untethered, real-time processing and monitoring; a data logging device for long-term ambulatory recording; a DSP integrated circuit for real-time processing of the signals locally; or a direct cable connection to a computing device for a minimum power, high reliability connection.

The ability to reconfigure the input and output of the system based on application requirements is an important feature of the system. The preferred embodiment features a wireless Bluetooth transceiver. In this configuration the system is used primarily for real-time signal-processing research and investigation into brain-computer interfaces. Another application could use the system as an EEG “Holter Monitor” by configuring the system with a data logging output module. By configuring the IO module with a DSP IC, alertness algorithms could be run in real-time and if the wearer's alertness drops off, the host node would activate a speaker in the system to alert the user.

The design of the EEG-specific data node contains numerous improvements on current EEG acquisition systems. A novel electrode assembly is incorporated into the EEG data nodes that address common problems in electrode application. Traditionally, applying electrodes to the scalp requires either gluing the electrodes to shaved patches of the head or wearing a tight-fitting elasticized cap with electrodes sewn in preset locations. In the latter case, for good ionic conduction, a large amount of electrolytic gel must be injected between the electrode and the scalp to saturate all the interfering hair. The invention's electrode assembly as depicted in FIGS. 7,8 addresses these limitations.

The electrode assembly designed for the EEG data nodes has superior positioning and hair penetrating capability when compared with traditional methods. The electrode assembly is positioned with two degrees of freedom by sliding the data node in one direction along its supporting distribution rail, and then positioning this distribution rail in an orthogonal direction with respect to medial rail to which it is attached. The assembly provides a third degree of freedom by extending or retracting the electrode-carrying probe towards or away from the scalp. To establish firm contact with the scalp, the assembly contains a low-impedance spring-loaded probe with a biopotential electrode at the end. The entire assembly is telescoped in and out by a control knob. The ability to twist the electrode allows a technician to work the electrode through masses of hair down to the scalp, thereby establishing the best possible contact without forcing the user to shave patches of hair. Additionally, the spring force of the electrode assembly maintains solid contact between the electrode and the scalp and also provides a restoring force to the frame in the event of disturbance.

In additional to the novel design of the electrode assembly, the treatment of the signal collected by the electrode is unique. The signal is passed directly from the electrode to the node's onboard circuitry for amplification and digitization. The total travel of the signal over unshielded conductor from the electrode to the data node's internal electronics is less than 10 mm, and may be less than 3 mm. Once inside the node housing, the analog signal from the electrode is immediately amplified, conditioned and converted to digital information. This immediate amplification and digitization of the signal at the source provides a substantial improvement in noise immunity over the electrodes and long leads now being used for EEG signal acquisition. Although systems currently exist that incorporate buffering or amplification circuits near the electrode, the resulting signal is then passed over a standard, and often quite long, length of wire. These leads are not shielded and are still very noise susceptible. An added drawback to these arrangements results from the fact that active electrode requires power, reference and signal wires into the electrode, thus requiring five leads rather than only one, which exacerbates wire management problems.

Additional functionality can be incorporated into data nodes by including a microcontroller in the node. The EEG data nodes of the preferred embodiment use a microcontroller for the A/D conversions, to interface with the serial communications bus, to control the gain of the amplifiers, and to control light emitting diodes (LEDs) that display status indications. Additional “intelligent” features can be implemented in the microcontroller. For example, the microcontroller could monitor its own data and send an alert to the host node if the electrode loses contact.

The mechanical design of the nodes and distribution rails is also an important feature of the preferred embodiment. The system of sliding nodes and rails allows nearly limitless reconfiguration of the system. The system is designed to support an entire 10-20 electrode placement system (described below), but it does not have to be used in this full configuration. If, for example, an application is known to only require a two-electrode setup, the system can be easily configured with a minimal set of nodes and rails to acquire these signals. The unconstrained sliding of the nodes and rails also allows a user to cluster sensors at locations of interests. For example, multiple EEG data nodes can be clustered with multiple DOT emitters and receivers to collect highly focused data from one or more locations on the cortex.

The design of the invention also offers superior electrostatic shielding. The distribution rails and node housings are all machined out of high-quality aluminum. Aluminum was chosen for its resistance to corrosion and high strength to weigh ratio, but it is also a very good conductor. By grounding the aluminum frame to the common ground on the ear, the frame becomes an extensive, low-impedance shield for the entire system. This shielding serves to minimize emissions from the electronics but also blocks external radiation from coupling to sensitive signal lines.

Applications

All these features combine to make a robust brain-sensing interface that is lightweight, portable, wireless, and flexible. This innovation opens up vast areas of application that were, until now, inaccessible. The immediate applications of the technology are in areas that have traditionally been limited by the cumbersome nature of existing clinical EEG setups. These applications include long-term or ambulatory monitoring of patients for diagnosis or assessment. Possible application areas along this line are long-term stroke assessment and rehabilitation, ADHD diagnosis and treatment, coma monitoring, and ambulatory seizure onset monitoring. In addition, the numerous advantages of the system also make it an attractive replacement for most clinical applications that use standard EEG monitoring equipment.

The system contemplated by the invention may be used to implement the internationally standardized 10-20 system usually employed to record spontaneous EEG. In the 10-20 system, twenty one electrodes are located on the surface of the scalp in positions are determined by an established protocol standardized by the American Electroencephalographic Society as described by the American Electroencephalographic Society Guidelines for Standard Electrode Position Nomenclature. J. Clin. Neurophysiol 8: 200-2. (Sharbrough et al., 1991). More recent guidelines for EEG-recording are published in the American Electroencephalographic Society guidelines in electroencephalography, evoked potentials, and polysomnography, J. Clin. Neurophysiol. 11:(1, January) (Gilmore RL (1994).

The invention may further be used to sense brain activity using diffuse optical tomography (DOT). DOT is a emerging imaging technology in which tissue is illuminated by near-infrared light from an array of emitters and the scattered light, some of which takes a path into the tissue and is then back-scattered to the surface near the point of emission (on average, a “banana-shaped” path from the point of entry to the point of exit), is measured by an array of photo-sensitive detectors on the surface of the tissue. The technology takes advantage of the fact that, at near-infrared wavelengths, the interfering tissues, skin and bone, are relatively transparent; additionally, the primary light absorbing molecules (chromophores) at these wavelengths are water, and two important metabolic markers of brain activity: oxyhemoglobin (HbO2) and deoxyhemoglobin (HbR). This allows hemodynamic changes resulting from brain activity to be assessed effectively and non-intrusively.

The most cost-effective and easily miniaturized DOT measurement technique is known as “continuous-wave.” In this type of system, light sources emit continuously (or with slow modulation for synchronous detection), and the change in amplitude at the detector is correlated to increases or decreases in chromophore concentration. Currently, continuous-wave techniques are the most suitable for integration into data nodes, though future advances could make other techniques equally viable.

When incorporated into the invention's node based architecture, each light source becomes an emitter node that irradiates the tissue with light from at two near-infrared LEDs of separate wavelengths. Using distinct wavelengths allows one to distinguish the relative absorption of each chromophore by taking advantage of their absorption characteristics at different frequencies. The back-diffused light is then collected nearby by a detector node equipped with a sensitive photodiode and related amplification, demodulation, and conditioning, circuitry.

Correct emitter/detector separation is essential to achieving good results in DOT systems; too much separation gives unacceptable signal attenuation, while too little separation collects photons that have not penetrated deep enough to contain useful information. Sliding nodes along rails allows one to easily find the optimal emitter/detector separation that gives optimal cortical penetration.

Further considerations when designing nodes for DOT include: optical isolation of detectors from the emitters and the environment; implementation of synchronous detection to improve stray-light rejection; and component selection for maximum dynamic range.

Data nodes may also used for electrocorticography, a technique of recording the electrical activity of the cerebral cortex by means of electrodes placed directly on it. Implanted electrocorticography electrodes may be connected by short lead wires to an adjacent data node, and implanted and contact peripheral electrodes may stimulated on command from a host node so that motor and sensory systems of the cortex may be mapped. This mapping may be use to help determine the location of eloquent brain matter, and therefore where the surgeons should not cut during surgery.

In addition to clinical applications for monitoring brain activity, many accessibility applications will be enabled by the portability of the system. These applications include interfaces for locked-in patients to facilitate communication, environmental control, and mobility. Embedded electrodes may be placed in the nodes to facilitate direct brain control over assistive robotic technologies.

Other immediate applications for the invention are in the brain sciences. Here the invention can be applied to research in neurotherapy, attention, performance, sources and triggers for emotions, expectation and intent, etc. The ability of the system to incorporate EEG and DOT modalities will facilitate research that looks at correlating neuronal activity with metabolic activation of cortical areas. This functionality may also be used for motor control and performance research.

The invention may also be used outside the medical and research arenas. In the consumer electronics industry, for example, the device may be used to provide brain control over remote-controlled cars or planes. Similar devices, with the appropriate configuration and algorithms, could be used to control household appliances and home electronics.

The invention can be used for “natural reaction assessment” when used in conjunction with algorithms for extracting emotional response from brain signals. This area of application takes advantage of the ability to determine, in real-time, a person's emotional disposition, i.e. whether they are happy, sad, agitated, bored, alert, or frightened. In the entertainment industry, the device could be used by participants in a movie audience to provide feedback on the quality of a film. For example, it might be able to provide information on what percentage of the audience felt bored during particular sections during the screening of the film. This information could then be used to re-edit the film and improve the product before release. A similar device could be used in the classroom to provide important feedback to the teacher on students' attentiveness and comprehension during lessons. A variation on the system could monitor and record a person's emotional state throughout the day. This information could be used in wellness applications or for therapy purposes.

The invention may also be used for lie detection. Using current and emerging algorithms in conjunction with the invention, a highly reliable lie detector may be implemented for use in courtrooms, police stations, and other places.

The invention may be used to implement an alertness monitor by incorporating algorithms for alertness and attention into a DSP module that would sound an audible alarm if the user's alertness drops below a critical level. These alertness monitors could then be used by pilots, drivers, air-traffic control workers, or anyone else whose job demands long, sustained periods of attention.

It is to be understood that the specific embodiments and applications that have been described are merely illustrative implementations of the principles of the present invention. Numerous modifications may be made to the arrangements and methods which have been described without departing from the true spirit and scope of the invention.

### CONCLUSION

It is to be understood that the methods and apparatus which have been described above are merely illustrative applications of the principles of the invention. Numerous modifications may be made by those skilled in the art without departing from the true spirit and scope of the invention.

1. A system for acquiring data indicating brain activity comprising, in combination,
a mechanical support frame adapted to be worn on a human head consisting of a plurality of mechanically connected rails, each of said rails including at least one data distribution conductor extending along substantially the entire length of said rail, the data distribution conductors in all of said rails being electrically connected together to form a data transmission network,
at least one data acquisition node mounted on one of said rails, said node data node including a sensor for acquiring data indicating brain activity from a localized area on said human head, said data node including means for transmitting said data via said power distribution conductor, and
a host node mounted on one of said rails for receiving data via said data distribution network for said data acquisition node and for relaying said data to an external utilization device.

2. A system for acquiring of data indicating brain activity as set forth in claim 1 wherein each of said rails in said support frame further includes a source of electrical power and a power distribution conductor extending along substantially the entire length of each of said rails, the power distribution conductors in said rails being electrically connected together and to said source of electrical power to form a power distribution network.

3. A system for acquiring of data indicating brain activity as set forth in claim 1 wherein said data node is mounted for adjustable movement along the rail upon which it is mounted and includes means for mechanically securing said data node in place at a desired position on said rail upon which it is mounted.

4. A system for acquiring of data indicating brain activity as set forth in claim 3 further including distribution nodes for mechanically connecting rails at an intersecting position, each of said distribution nodes being mounted for adjustable movement along one of said rails.

5. A system for acquiring of data indicating brain activity as set forth in claim 4 wherein said sensor is mounted on a probe mounted on said data node for movement toward and away from said human head.

6. A system for acquiring of data indicating brain activity as set forth in claim 5 wherein said sensor comprises a biopotential electrode adapted to establish electrical contact with the human skin.

7. A system for acquiring of data indicating brain activity as set forth in claim 6 wherein said data acquisition node includes means for resiliently forcing said biopotential electrode against the skin.

8. A system for acquiring of data indicating brain activity as set forth in claim 6 wherein said sensing probe extends through said data acquisition node and includes a biopotential electrode on the end adjacent to said human head and a control knob at the other end which permits said sensing probe to be twisted to establish better contact between said biopotential sensor and said human head.

9. A system for acquiring of data indicating brain activity as set forth in claim 1 where data node includes an analog-to-digital converter for converting data acquired by said sensor into digital data transmitted via said distribution network to said host node.

10. A system for acquiring of data indicating brain activity as set forth in claim 9 where digital data is serialized for transmission over two wires in said distribution network.

11. A system for acquiring of data indicating brain activity as set forth in claim 9 where each of said rails contains two or more parallel signal carrying conductors for simultaneously transmitting different data quantities.

12. A system for acquiring of data indicating brain activity as set forth in claim 1 wherein each of said rails further includes a reference signal conductor and system further comprises a reference node mounted on one of said rails for connecting said reference signal conductor to an external object.

13. A system for acquiring of data indicating brain activity as set forth in claim 1 where host node includes a radio communications transceiver for transmitting said data to an external utilization device.

14. A system for acquiring of data indicating brain activity as set forth in claim 1 wherein said intersecting rails are curved such that said support frame forms a helmet that defines an anatomically shaped cavity that surrounds and substantially conforms to the shape of said human head.

15. A system for acquiring of data indicating brain activity as set forth in claim 1 wherein said data acquisition node acquires electroencephalography data.

16. A system for acquiring of data indicating brain activity as set forth in claim 15 further comprising a plurality of further data acquisition nodes mounted on said rails.

17. A system for acquiring of data indicating brain activity as set forth in claim 16 wherein at least some of said further data acquisition nodes acquire electroencephalography data.

18. A system for acquiring of data indicating brain activity as set forth in claim 16 wherein at least some of said further data acquisition nodes acquires optical tomography imaging data.

19. A system for acquiring of data indicating brain activity as set forth in claim 16 wherein at least some of said further data acquisition nodes acquire electrocorticography data.

20. A system for acquiring of data indicating brain activity as set forth in claim 1 wherein said data acquisition node includes and acquires optical tomography imaging data.

21. A system for acquiring of data indicating brain activity as set forth in claim 1 wherein said data acquisition node acquires electrocorticography data.

22. A system for acquiring of data indicating brain activity as set forth in claim 1 wherein said data acquisition node includes a sensor for producing an analog electrical signal whose amplitude is indicative of brain activity and an amplifier having an input connected to said sensor for amplifying said analog signal.

23. A system for acquiring of data indicating brain activity as set forth in claim 22 wherein said amplifier is located less than 10 millimeters from said sensor.

24. A system for acquiring of data indicating brain activity as set forth in claim 1 wherein said data acquisition node includes a sensor for producing an analog electrical signal whose amplitude is indicative of brain activity and an analog-to-digital converter for producing a digital output signal having a value representing said amplitude for transmission via said data transmission network.

25. A system for acquiring of data indicating brain activity as set forth in claim 24 wherein said analog-to-digital converter is located less than 10 millimeters from said sensor.

26. A system for acquiring of data indicating brain activity as set forth in claim 1 wherein said rails are constructed of an electrically conductive material which provides shielding for said data distribution conductors.

27. A system for acquiring of data indicating brain activity as set forth in claim 26 wherein said electrically conductive material is aluminum.

28. A system for acquiring data indicating brain activity comprising, in combination,
a rigid helmet defining an interior cavity shaped to conform to and be worn on a human head,
a plurality of data acquisition sensors attached to said helmet at adjustable spaced apart locations thereon, each of said sensors including a sensing element which extends inwardly from said helmet to a selected position on or near said human head,
and a radio transceiver mounted on said helmet for transmitting data acquired by said sensors to a remote data utilization device.

29. A system for acquiring data indicating brain activity as set forth in claim 28 wherein at least one of said data acquisition sensors is an electrode for establishing an electrically conductive contact with the skin at said selected position for acquiring data in the form of a biopotential signal.

30. A system for acquiring data indicating brain activity as set forth in claim 29 wherein at least one of said data acquisition sensors acquires optical tomography imaging data from said selected position on or near the human head.

31. A system for acquiring data indicating brain activity as set forth in claim 29 wherein said one of said data acquisition sensors further includes a resilient member for urging said electrically conductive contact against the skin.

32. A system for acquiring data indicating brain activity as set forth in claim 28 wherein at least one of said data acquisition sensors acquires optical tomography imaging data.

33. A system for acquiring data indicating brain activity as set forth in claim 28 wherein said radio transceiver transmits data acquired by said sensors in digital form to said remote data utilization device.

34. Apparatus for acquiring brain activity data comprising, in combination,
a rigid helmet anatomically shaped to conform to the human head of a wearer,
a plurality of data acquisition sensors attached to an adjustable location on said helmet, each of said sensors including a probe mounted for movement toward and away from said human head to position a sensing element at or near a selected position on the head, and
a communications unit attached to said helmet for receiving sensing data from each of said sensors and for transmitting said sensing data to a utilization device external to said helmet.

35. Apparatus for acquiring brain activity data as set forth in claim 34 wherein at least one of said data acquisition sensors is an electrode for establishing an electrically conductive contact with the skin at said selected position for acquiring data in the form of a biopotential signal.

36. Apparatus for acquiring brain activity data as set forth in claim 35 wherein said one of said data acquisition sensors further includes a resilient member for urging said electrically conductive contact against the skin.

37. A system for acquiring data indicating brain activity as set forth in claim 34 wherein at least one of said data acquisition sensors acquires optical tomography imaging data.

38. A system for acquiring data indicating brain activity as set forth in claim 34 wherein said radio transceiver transmits data acquired by said sensors in digital form to said remote data utilization device.
